(19) United States
(12) Patent Application Publication
(10) Pub. No.: US 2021/0000104 A1
(43) Pub. Date: Jan. 7, 2021

(54) Methods, systems and apparatus for preservation of organs and other aqueous-based materials utilizing low temperature and elevated pressure

(71) Applicant: CryoStasis Ltd., Westport (CA)

(72) Inventors: Olga Kukal, Westport (CA); Thomas Furman Allen, Westport (CA); Bill Russell Alexander, Shrewsbury, MA (US)

(21) Appl. No.: 16/501,918

(22) Filed: Jul. 5, 2019

(51) Int. Cl. A01N 1/02

(52) U.S. Cl. CPC A01N 1/0284; A01N 1/0252; A01N 1/021; A01N 1/0289

(57) ABSTRACT

This invention uses low temperature and elevated pressure to induce suspended animation by depressing the freezing and melting temperature of water and aqueous solutions, including but not limited to biological materials, soluble molecules, organic and inorganic compounds. Increasing the pressure to ˜210 MPa in a container depresses the freezing and melting temperature of water, biological matter, and materials in aqueous solution, to ˜−22° C. Storage at low temperature under high pressure suspends metabolic activity and induces cryostasis. This invention can be used for cryo-banking biological materials that cannot be frozen or vitrified, or otherwise preserved, including, but not limited to, cells, tissues, human organs for transplantation or entire organisms.

### US Patent Documents

| | | |
|---|---|---|
| 3,406,531 | October 1968 | Swenson et al. |
| 3,468,136 | September 1969 | Swenson et al. |
| 3,545,221 | December 1970 | Swenson et al. |
| 3,607,646 | September 1971 | de Roissart, H |
| 3,632,473 | January 1972 | Belzer, et al. |
| 3,654,085 | April 1972 | Fritz et al. |
| 3,772,153 | November 1973 | de Roissart, H |
| 4,186,565 | February 1980 | Toledo-Pereyra, L |
| 4,559,298 | August 1985 | Fahy, G |
| 4,605,644 | August 1986 | Folker, J |
| 4,688,387 | August 1987 | Conway, R |
| 4,798,824 | January 1989 | Belzer, et al. |
| 4,837,390 | June 1989 | Reneau, R |
| 4,873,230 | October 1989 | Belzer, et al. |
| 4,879,283 | November 1989 | Belzer, et al. |
| 5,051,352 | September 1991 | Martindale, et al. |
| 5,145,771 | September 1992 | Lemasters, et al. |
| 5,200,398 | April 1993 | Strasberg, et al. |
| 5,217,860 | June 1993 | Fahy, et al. |
| 5,306,711 | April 1994 | Andrews, P |
| 5,358,931 | October 1994 | Rubinsky, et al. |
| 5,370,989 | December 1994 | Stern, et al. |
| 5,472,876 | December 1995 | Fahy, G |
| 5,586,438 | December 1996 | Fahy, G |
| 5,709,654 | January 1998 | Klatz, et al. |
| 5,723,282 | March 1998 | Fahy, et al |
| 5,752,929 | May 1998 | Klatz, et al. |
| 5,756,547 | May 1998 | Horwitz, L |
| 5,804,444 | September 1998 | Kukal, et al. |
| 5,856,081 | January 1999 | Fahy, G |
| 5,952,168 | September 1999 | Wowk, et al. |
| 5,962,214 | October 1999 | Fahy, et al. |
| 6,024,698 | February 2000 | Brasile, L |
| 6,045,990 | April 2000 | Baust, et al. |
| 6,046,046 | April 2000 | Hassanein, W |
| 6,187,529 | February 2001 | Fahy, et al. |
| RE37,892 | October 2002 | Kukal, et al. |
| 6.492,103 | December 2002 | Taylor, M |
| 6,519,953 | February 2002 | Kukal, et al |
| 6,569,615 | May 2003 | Thatte, et al. |
| 6,616,858 | September 2003 | Fahy, et al. |
| 6,673,594 | January 2004 | Owen, et al |
| 6,677,150 | January 2004 | Alford, et al. |
| 6,736836 | May 2004 | Montgomery, R |
| 6,740,484 | May 2004 | Khirabadi, et al. |
| 6,803,227 | October 2004 | Wood, et al. |
| 6,869,757 | March 2005 | Fahy, G |
| 6,921,633 | July 2005 | Baust, et al. |
| 6,977,140 | December 2005 | Owen, et al. |
| 6,994,954 | February 2006 | Taylor, M |
| 7,029,893 | April 2006 | Toledo-Pereyra, et al. |
| 7,157,222 | January 2007 | Khirabadi, et al. |
| 7,316,922 | January 2008 | Streeter, J |
| 7,476,660 | January 2009 | Masaki, et al. |
| 7,572,622 | August 2009 | Hassanein, et al. |
| 7,678,563 | March 2010 | Wright, et al. |
| 7,713,686 | May 2010 | Poo, et al. |
| 7,741,018 | June 2010 | Fahy, G |
| 7,790,437 | September 2010 | Poo, et al. |
| 7,837,895 | November 2010 | Fujikawa, et al. |
| 7,993,681 | August 2011 | Motterlini, et al. |
| 7,998,182 | August 2011 | Dobak III, et al. |
| 8,097,449 | January 2012 | Garland, et al. |
| 8,211,628 | July 2012 | Thatte, et al. |
| 8,268,547 | September 2012 | Owen, et al. |
| 8,268,612 | September 2012 | Owen, et al. |
| 8,288,084 | October 2012 | Lee, H |
| 8,318,415 | November 2012 | Owen, et al. |
| 8,323,954 | December 2012 | Kravitz, et al. |
| 8,320,450 | December 2012 | Faulkner, et al. |
| 8,354,221 | January 2013 | Roy, et al. |
| 8,389,271 | March 2013 | Wright, et al. |
| 8,394,624 | March 2013 | Kilic, et al. |
| 8,409,846 | April 2013 | Hassanein, et al. |
| 8,420,380 | April 2013 | Fishman, et al. |
| 8,420,381 | April 2013 | Owen, et al |
| 8,431,385 | April 2013 | Owen, et al |
| 8,445,260 | May 2013 | Owen, et al |
| 8,465,970 | June 2013 | Hassanein, et al. |
| 8,535,394 | September 2013 | Hassanein, et al. |
| 8,585,380 | November 2013 | Hassanein, et al. |
| 8,679,735 | March 2014 | Fahy, et al. |
| 8,748,368 | June 2014 | Cicardi, et al. |
| 8,753,806 | June 2014 | Mangino, et al. |
| 8,822,203 | September 2014 | Hassanein, et al. |
| 8,828,710 | September 2014 | Anderson, et al. |
| 8,834,399 | September 2014 | Muller-Spanka, et al |
| 8,900,804 | December 2014 | Kamlot, et al. |
| 8,927,257 | January 2015 | Hutzenlaub, et al. |
| 8,945,823 | February 2015 | Thatte, et al. |
| 8,962,303 | February 2015 | Owen, et al. |
| 9,055,740 | June 2015 | Hassanein, et al. |
| 9,089,126 | July 2015 | Faulkner, et al. |
| 9,215,867 | December 2015 | Hassanein, et al. |
| 9,247,729 | February 2016 | Patrick III, et al. |
| 9,253,976 | February 2016 | Anderson, et al. |
| 9,258,995 | February 2016 | MacMilliam-Crow |
| 9,301,519 | April 2016 | Hassanein, et al. |
| 9,320,269 | April 2016 | Brassil, J |
| 9,506,846 | November 2017 | Rubner, et al. |
| 9,706,769 | July 2017 | Taylor, et al. |
| 9,737,071 | August 2017 | Shimko, et al. |
| 9,756,849 | September 2017 | Hassanein, et al. |
| 9,756,850 | September 2017 | Hassanein, et al. |
| 9,756,851 | September 2017 | Hassanein, et al. |
| 9,789,133 | October 2017 | Wager, et al. |
| 9,867,368 | January 2018 | Anderson, et al. |
| 9,877,474 | January 2018 | Tsuji, et al. |
| 9,936,689 | April 2018 | Anderson, et al. |
| 9,955,682 | May 2018 | Kravitz, et al. |
| 10,039,277 | August 2018 | Cook, et al. |
| 10,076,112 | September 2018 | Hassanein, et al. |

### OTHER REFERENCES

Akasaka, K. and Matsuki, H. (Eds.) High Pressure Bioscience. Dordrecht: Springer, 2015. cited by applicant.

Becquerel, P. La suspension de la vie au dessous de 1/20 K absolu par demagnetization adiabatique de l'alun de fer dans le vide les plus eléve”[The suspension of life below 1/20 K absolute by adiabatic demagnetization of iron alum in the highest vacuum]. Comptes Rendus des Séances de l'Académie des Sciences (in French). 231 (4), 1950. cited by applicant.

Chapman, M. Water Structure and Science, 2018. http://www1.lsbu.ac.uk/water/water_structure_science.html cited by applicant.

Daucik, K. & Dooley, R. B. Revised release on the pressure along the melting and sublimation curves of ordinary water substance. The International Association for the Properties of Water and Steam, IAPWS R14-08, 2011. cited by applicant.

Dhen, et al. Practical Methods in Cardiovascular Research, Springer, NY, 2005. cited by applicant.

Fahy, G. M et al. Cryopreservation of organs by vitrification: perspectives and recent advances. Cryobiology, 2004. cited by applicant.

Franks, F. et al. Water A Comprehensive Treatise, Volume 1, The Physics and Physical Chemistry of Water, Plenum Press, NY, 1972. cited by applicant.

Franks, F. et al. Water A Comprehensive Treatise, Volume 2, Water in Crystalline Hydrates Aqueous Solutions of Simple Nonelectrolytes, Springer Science+Business Media, LLC., 1973. cited by applicant.

Franks, F. et al. Water A Comprehensive Treatise, Volume 3, Aqueous Solutions of Simple Electrolytes, Plenum Press, NY, 1973. cited by applicant.

Franks, F. et al. Water A Comprehensive Treatise, Volume 4, Aqueous Solutions of Amphiphiles and Macromolecules, Plenum Press, NY, 1978. cited by applicant.

Franks, F. et al. Water A Comprehensive Treatise, Volume 5, Water in Disperse Systems, Plenum Press, NY, 1975. cited by applicant.

Franks, F. et al. Water A Comprehensive Treatise, Volume 6, Recent Advances, Plenum Press, NY, 1979. cited by applicant.

Franks, F. et al. Water A Comprehensive Treatise, Volume 7, Water and Aqueous Solutions at Subzero Temperatures, Plenum Press, NY, 1982. cited by applicant.

Franks, F. Water, Royal Society of Chemistry, 1983. cited by applicant.

Franks, F. et al. Water Science Reviews 1, Cambridge University Press, UK, 1985. cited by applicant.

Franks, F. et al. Water Science Reviews 2, Cambridge University Press, UK, 1986. cited by applicant.

Franks, F. et al. Water Science Reviews 3, Cambridge University Press, UK, 1988. cited by applicant.

Franks, F. et al. Water Science Reviews 4, Cambridge University Press, UK, 1989. cited by applicant.

Franks, F. et al. Water Science Reviews 5, Cambridge University Press, UK, 1990. cited by applicant.

Franks, F. Biophysics and Biochemistry at Low Temperatures, Cambridge University Press, UK, 1985. cited by applicant.

Guibert et al. Organ preservation: Current concepts and new strategies for the next decade. Transfusion Medicine and Hemotherapy 38(2), 2011. cited by applicant.

Harned, H. S. & Owen, B. B. Physical Chemistry of Electrolytic Solutions, American Chemical Society Monograph Series, Reinhold Book Corporation, NY, 1958. cited by applicant.

Hochachka, P. W. & Somero, G. N. Biological Adaptations, Oxford University Press, NY, 2002 Toledo-Pereyra, L. H. Organ Preservation for Transplantation 3^{rd} Edition, Landes Bioscience, Austin Tex., 2010. cited by applicant.

Jonsson, K. I. et al. Tardigrades survive exposure to space in low earth orbit. Current Biology 18(7), 2008. cited by applicant.

Kanno, H. et al. Supercooling of water to −92° C. under pressure. Science 189(4206), 1975. cited by applicant.

Mazur, P. Organ Preservation for Transplantation. Dekker, N.Y., 1981. cited by applicant.

Ono et al. Effect of ultra-high pressure on small animals, tardigrades and Artemia. Cogent Physics 3(1), 2016. cited by applicant.

Pääbo et al. Genetic analyses from ancient DNA. Annual Review of Genetics 38(1), 2004. cited by applicant.

Seki, K. & Toyoshima, M. Preserving tardigrades under pressure. Nature 395(6705), 1998. cited by applicant.

Suzuki, K. & Taniguchi, Y. Effect of pressure on biopolymers and model systems. Symposia of the Society for Experimental Biology, 26, 1972. cited by applicant.

Weber, G., & Drickamer, H. G. The effect of high pressure upon proteins and other biomolecules. Quarterly Reviews of Biophysics 16, 1983. cited by applicant.

Walters, E. M. et al. The history of sperm cryopreservation In: Sperm Banking: Theory and Practice. Cambridge University Press, 2009. cited by applicant.

### STATEMENT REGARDING FEDERALLY SPONSORED RESEARH OR DEVELOPMENT

Research and development that culminated in this patent was not federally sponsored.

### FIELD OF THE INVENTION

This invention entails the methods for preservation of aqueous-based substances at low temperatures using elevated pressure to depress the freezing/melting temperature of water and/or aqueous substances. Initial application is the long-term storage, bio-banking, of human organs for transplantation.

### BACKGROUND OF THE INVENTION

This invention uses fields of medicine, biochemistry, thermodynamics and physical chemistry, and other applicable fields, in the development of new methods and devices for long-term preservation of aqueous-based materials, in particular medical devices for storage of biologically important substances, such as human organs. The current art involves perfusion and storage at body temperature or in the hypothermic range of 4° C. and above. These methods are efficacious for the preservation and transport of organs over several days. The technologies are not suitable for long-term bio-banking (weeks, months, years).

There is a growing need for transplantable organs, and countless people die each year waiting for an organ transplant. This situation can be partly ameliorated by improving the preservation of organs during transport, but these advances will only be incremental. Once regenerative technologies for 3-D printing, growing, and genetic/immunological modification of organs for xenotransplantation are realized, the need for transplantable organs can be met. The ability to print, grow, and/or genetically modify organs for transplantation will present new challenges. It will be necessary to store organs from these sources until needed for transplantation, because the processes used to manufacture the organs will take an interval of time that might not be available to a patient in critical need. Further, individuals may wish to have a set of their own organs/tissues generated and preserved for future needs.

This invention addresses the need for long-term storage and preservation of organs and other biological materials. The intellectual property is also suitable for, but not limited to, the long-term preservation of organic molecules, organelles, cells, tissues, organs, biologics, pharmaceuticals, and early studies indicate that it could be used to store entire organisms in a state of suspended animation, possibly facilitating interstellar travel. It has been documented that some molecules, cells and even organisms can tolerate extreme environmental conditions. The effect of pressure at ambient temperature on molecules, cells and organisms has been studied with results showing that survival is possible even at ultra-high pressures (Weber & Drickamer 1983; Seki & Toyoshima 1998; Ono et al. 2016). Some cells and organisms can remain viable at temperatures near absolute zero or in outer space (Becquerel 1950; Jonsson et al. 2008). Over more than half a century, researchers have attempted to develop methods of freezing or vitrifying organs as a means of long-term preservation (Mazur 1981; Fahy et al. 1990; Guibert et al. 2011). All attempts have met with failure. This invention provides a workable alternative to freezing as a means of long-term preservation of biological matter and other aqueous-based organic and inorganic materials.

### BRIEF SUMMARY OF THE INVENTION

The objective of this invention is to provide a solution to the problem of long-term preservation of biological materials, such as human organs. The solution is to avoid freezing (phase change) and maintain sensitive/unfreezable materials in a stable, liquid state at the lowest attainable temperature. This invention induces a state of molecular/physiological “stasis”, by means of elevated pressure, used to depress the freezing temperature (i.e. melting temperature) of water, biological matter, and other aqueous-based materials, both organic and inorganic. “Stasis” as it pertains to this invention is defined as “cryostasis”, a more accurate term, due to the low temperatures required to induce this state. The invention's methodology, employing pressure and temperature in concert, can facilitate long-term preservation (months, years) in cryostasis, and provide a means of bio-banking. The pressures involved can also induce a metastable, supercooled state that may be used for long-term preservation of aqueous-based materials. The invention utilizes the physicochemical properties of water, and its interactions with pressure and temperature, to maintain aqueous-based materials in a stable, liquid state. The preferred embodiment is, but not limited to, preservation at the lowest temperature and corresponding pressure at which water is in a stable, liquid state, with no possibility of freezing (please refer to the fusion curve (solid-liquid boundary) in FIG. 1. At the pressures essential to achieve the freezing/melting temperature depression, molecular motion and metabolism is suppressed, resulting in cryostasis.

### BRIEF DESCRIPTION OF THE DRAWINGS

FIG. 1: Phase Diagram of Water Showing the Relationship of Temperature and Pressure.

FIG. 1 depicts the relationship of pressure and temperature and the fusion curve (solid-liquid boundary) that delineates at which pressure/temperature values water remains in a stable, liquid state. The invention focuses on the lowest temperature and corresponding pressure at which water is in a stable, liquid form (designated as “A” in diagram). At temperatures below this lowest temperature and its corresponding pressure, water either supercools (undercools) or forms Ice III or Ice Ih. Likewise, at pressures above the pressure corresponding to the lowest temperature, water is metastable and can form Ice III or Ice Ih (A). These transition parameters pertaining to pressure and temperature define the coldest conditions that water, biological materials, and aqueous substances can remain in liquid state with no possibility of phase change.

FIG. 2: The Pressure-Temperature System

This drawing illustrates the major components of the invention. Elevated pressure is used to depress the freezing point of water in order to preserve aqueous-based material contained in the pressure vessel. The key parts of the system are described as follows:

01—Pressure generator hand-operated wheel

02—Pressure generator

03—Drive fluid isolation valve

04—Drive fluid reservoir

05—Pressure gauge (Analog)

06—Pressure transducer

07—Pressure generator isolation valve

08—Insulating cover of refrigerated compartment

09—Pressure vessel isolation valve

10—Refrigerated compartment

11—Cooler/heater

12—Temperature sensor for refrigeration control

13—Pressure vessel for storing material

14—Temperature sensor monitoring vessel interior

15—Circulating fan/stirrer

Please refer to a fuller description above in the “Detailed Description of the Invention” section.

FIG. 3: The Pressure Vessel for Preservation of Aqueous-Based Materials

The pressure vessel is used for containing material during long-term preservation. It is comprised of the following components.

01—Pressure vessel top

02—Retaining ring

03—O-ring seal

04—Pressure vessel body

### DETAILED DESCRIPTION OF THE INVENTION

Definitions and clarifications: Certain terminology used throughout the description of the invention requires clarification of meaning.

“Stasis” or “Cryostasis” is used to describe a state of suspended metabolic and molecular activity. Cryostasis pertains more specifically to the sub-zero ° C. temperature and pressure range described in the invention.

“Suspended animation” pertains to as state of inactivity similar to that described above.

“Material”, “substance”, “matter” are terms used interchangeably in the description. They refer to either biological or inorganic constituents that are difficult to preserve over long-term period.

“Biological material” refers to carbon-containing, living matter or previously viable matter, including but not limited to molecules, cells, organelles, tissues, organs, organisms.

“Aqueous-based material” is a general term for any organic or inorganic matter that is soluble in water, or suspended in water, or contains water.

“Sub-zero” temperature is used in reference to storage at any temperature below 0° C.

“Banking” or “bio-banking” applies to the long-term preservation and storage of either biological or inorganic material.

“Storage” or “preservation” are terms used interchangeably throughout the description, and refer to the conservation and maintenance of material in cryostasis.

“Device(s)” or “apparatus” are assumed to be in plural case unless specified otherwise.

“Preferred embodiment”: Unless specifically referred to as the preferred embodiment, all of the embodiments described in this invention are general in nature.

“˜”: All numbers except those describing the preferred embodiment are approximate and not limited to the precise numeral stated.

The singular forms “a”, “an” and “the” include plural referents unless clearly stated otherwise.

“Fluid” refers to a gas, liquid, or a combination thereof, unless clearly specified.

“Supercooled” or “undercooled” refers to the metastable state of water below its melting temperature of 0° C. and atmospheric pressure.

“Colligative” depression of the melting (freezing) temperature of water is defined by the number of molecules in solution. One mole of solute dissolved in 1 litre of water results in 1.86° C. melting point depression.

“Non-colligative” depression of the melting (freezing) temperature of water is achieved through ice inhibiting or ice binding agents that prevent, inhibit, control, and/or sequester ice crystal growth.

“Long-term” as pertains to this invention describes any time period from weeks and months to years, unless specifically stated.

“Freezing point depression” (FPD) refers to the lowering of melting (freezing) temperature of water below 0° C. It can be achieved as described in this document through increase in pressure, supercooling, and/or addition of colligative or non-colligative acting substance.

Preservation of aqueous-based substances and biological materials sensitive to cryoinjury and/or freezing has been a dilemma. Some molecules (e.g. DNA), cells (e.g. bovine spermatozoa) and organisms (e.g. tardigrades, brine shrimp) can be successfully stored frozen for years (Pääbo et al. 2009; Walters et al. 2009; Ono et al. 2016). However, most biological substances (e.g. mammalian organs) cannot survive freezing or long-term storage. The reasons for this are multifold and relatively well understood (Guibert et al. 2011). For instance, the ˜9% increase in volume during phase change from liquid to solid water (Ice Ih) causes physical damage to membranes, cells and molecular machinery. This damage is exacerbated by cell dehydration as a result of osmotic imbalance, and recrystallization of ice during the thawing process. Rapid, uniform rates of freezing are difficult, if not impossible, to achieve for biological substances that have volumes that are numerically (dimentionless) greater than their surface areas, such as human organs. In these cases, freezing starts rapidly from the outside, and when the interior later freezes, it expands and ruptures the exterior layers, hence causing physical damage. This invention is aimed to circumvent the inherent problems with phase change between liquid and solid by preventing it. A new system has been devised to prevent phase change, where water and aqueous-based substances can be maintained over long-term intervals in a stable, liquid state, at temperatures below their melting point at atmospheric pressures by means on increased pressure.

The current invention is based on the hypothesis: The colder biological and other aqueous-based materials are stored without freezing and thawing (phase transition), the longer they will remain in usable (functional) condition (i.e., the lower the storage temperature, the longer the viable storage duration). Hence the question arises: Can temperature of living matter be lowered sufficiently without freezing in order to induce cryostasis? For example, mammalian cells, tissues, organs, and organisms are aqueous-based with approximately 300 millimoles of dissolved solutes. Based on colligative properties, these 300 millimoles of solutes result in a 0.55° C. freezing point depression of the solution within mammalian tissues. A storage temperature of —0.55° C. is not low enough to sufficiently extend (i.e. only by hours, not even days) the usable life of an organ for transplantation. In order to achieve storage at temperatures low-enough to preserve cells, tissues, organs, and organisms for months or years an alternative methodology is needed. The key to this methodology lies in the relationship of temperature to pressure.

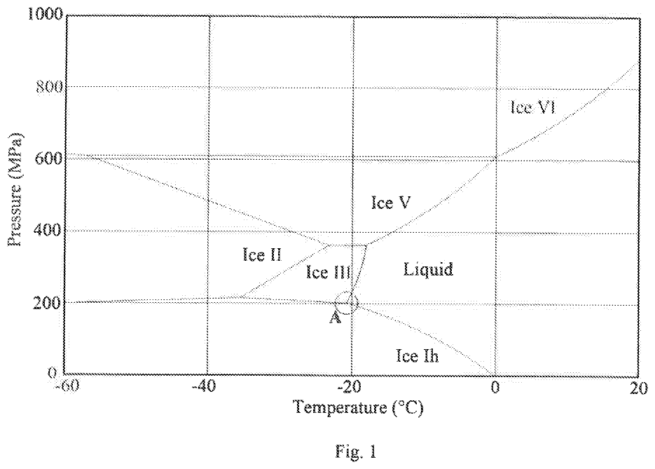

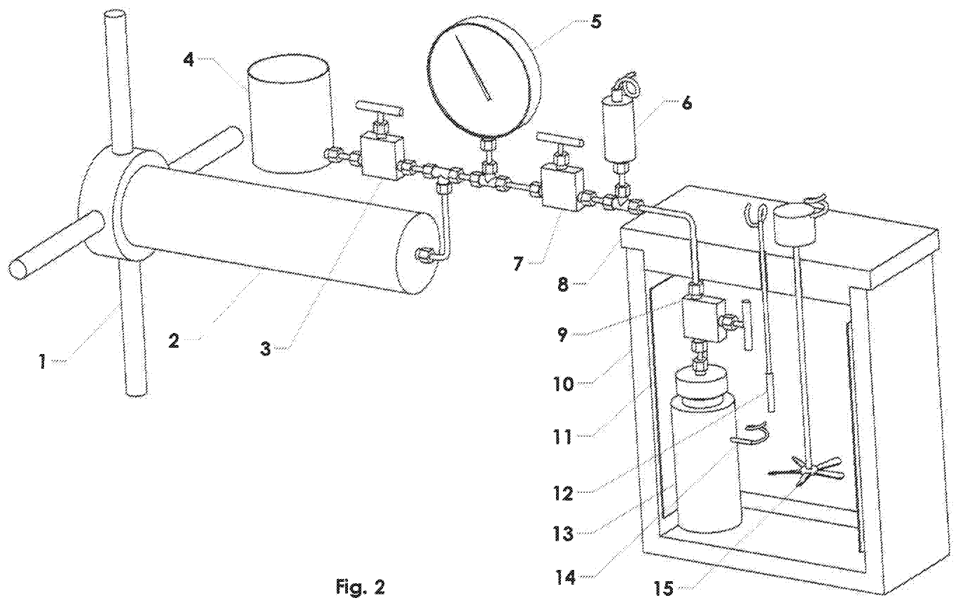

This invention uses elevated pressure (i.e. above ambient, atmospheric pressure) to depress the freezing/melting point of water and aqueous solutions. The freezing point of pure water, and thus all aqueous-based and biological material, can be depressed by ˜1° C. per ˜9.5 MPa (Daucik & Dooley 2011). For instance, pressure of ˜210 MPa lowers the freezing point of water and aqueous solutions to ˜−22° C. (refer to FIG. 1). Under these environmental conditions, molecular motion is reduced to the point that metabolic function is suppressed, resulting in a state of suspended animation, which the inventors term “cryostasis”. Cells, tissues, organs, and organisms stored under high pressure/low temperature conditions for days to weeks to months do not show signs of deterioration, apoptosis or necrosis and retain their functionality (Table 1). Using the environmental conditions described above the maximum storage interval for biological substances and other aqueous-based materials is yet to be determined and may well have no tangible temporal limit.

Broadly stated, this invention provides a means for storing biological and aqueous-based materials, unfrozen below 0° C., by means of pressure elevated above ambient pressure.

Biological and aqueous-based substances stored under ˜210 MPa of pressure and at a temperature no lower than ˜−22° C. will remain in a stable liquid state, because as pressure increases, the melting/freezing point of water decreases. FIG. 1 depicts the relationship of pressure and temperature and the fusion curve (solid-liquid boundary) that delineates at which pressure/temperature values water remains in a stable, liquid state. The invention focuses on the lowest temperature and corresponding pressure at which water is in a stable, liquid form. At temperatures below this lowest temperature and its corresponding pressure, water either supercools (undercools) or forms Ice III or Ice Ih (see FIG. 1, “A”). Likewise, at pressures above the pressure corresponding to the lowest temperature for stable liquid water, water is metastable and can form Ice III or Ice Ih. These critical point parameters pertaining to pressure and temperature define the coldest conditions that water, biological materials, and aqueous substances can remain in liquid state with no possibility of freezing (phase change). Preserving biological material such as cells, tissues, organelles, molecules, organs, and/or organisms under environmental conditions of elevated pressure (above atmospheric) and temperatures below the freezing temperature of water (i.e. melting temperature) at atmospheric pressure (Earth's surface), supresses enzymatic and overall metabolic activity. As temperature decreases and pressure increases this suppression transitions into cryostasis, a state of suspended animation with virtually no metabolic activity. This invention embodies a means of storing biological and other aqueous-based materials in a state of suspended animation, i.e. cryostasis. The suspension of metabolism (aerobic and anaerobic), apoptosis and/or necrosis during cryostasis provides for the long-term preservation (i.e. banking) of organic and inorganic aqueous-based materials. The lower the storage temperature, the greater the depth of the state of stasis.

Preservation of aqueous-based materials in a non-frozen state can be extended beyond the above described use of pressure to depress the freezing (melting) point to ˜−22° C. There are three means of achieving further freezing point depression (FPD):
1) Supercooling: Aqueous-based materials can be supercooled under pressure where a metastable liquid state can be maintained to at least −92° C. (Kanno et al. 1975).
2) Colligative freezing point depression: Addition of soluble substances to water further depresses the freezing point of the solution below ˜−22° C. under ˜210 MPa. The additional FDP will be equal to 1.86° C. per each mole of colligatively acting solute.
3) Non-colligative freezing point depression: Non-colligative agents provide an additive freezing point depression by means of ice inhibiting or ice binding agents, thus preventing, inhibiting, controlling, and/or sequestering ice crystal growth.

The three methods described above can be used individually or in concert to lower the storage temperature of unfrozen materials below ˜−22° C. under ˜210 MPa. Employing these techniques will extend preservation time for materials requiring cryostasis.

The environmental conditions for storage at or near pressure of ˜210 MPa and temperature of or near ˜−22° C. require a pressure vessel, and a device capable of generating pressure to pressurize and de-pressurize a pressure vessel. A vessel capable of containing these pressures without failing may be comprised of steel, stainless steel, titanium, or some other appropriate material. The vessel needs to have a means of loading and removing the material stored, and a means of connecting the pressure generator to the vessel. The pressure generator (hydraulic, pneumatic, but not limited to either) can be operated manually, using a timer to control the rate of pressurization and de-pressurization. Alternatively, the pressure generator can be automated and driven mechanically, pneumatically or hydraulically, or by other means, and controlled by an electrical, electronic, computer or mechanical analog, or other controller. The preferred embodiment is a hydraulic pressure generator (see section below on the preferred embodiment).

The preferred means of connecting the pressure generator to the pressure vessel is, but not limited to, by a system of pipes, valves, junctions, fittings, pressure gauge(s) and hydraulic fluid reservoir. In order to decrease or increase temperature of the pressure vessel a controlled cooling and heating system is required. The heat transfer medium can be either fluid or solid. For example, in the case of a cooling/heating system using fluid as the heat transfer vehicle, a container is required to contain the medium, supplied with either a cooler/heater. The heater can be separate from the cooler with its own temperature sensor and temperature controller, or they can be integrated.

A temperature controller controls the cooler/heater by means of temperature data provided by a temperature sensor immersed in the fluid and/or inserted into the pressure vessel. The temperature controller can either be computer software, or a stand-alone controller, or other means of control. The sensor can be a thermocouple, thermistor, RTD (Resistance Thermal Device), or any other appropriate device.

A cooling/heating system using fluid as the heat transfer medium requires a mixing unit, or some other device, to provide constant mixing of the transfer media. Mixing is important for efficient, and better-controlled method of heat transfer, enabling uniform temperature throughout the fluid enclosure, and preventing thermoclines. A pressure gauge, or other measuring/monitoring device, is used to monitor pressure. This can either be, but not limited to, an analog gauge or a pressure transducer connected to a display, or a data acquisition system (DAQ) attached to a computer that displays and records the pressure. Temperature of the fluid in the enclosure and/or of the interior of the pressure vessel is monitored with temperature sensors (thermocouples, thermometers, thermistors, RTDs or other suitable device(s)), and data strings are displayed and/or recorded by means of a DAQ/computer system, or other method/system. A thermometer, or other temperature sensor, can be immersed or partially immersed in the fluid to monitor temperature. The pressure vessel remains in the fluid during cooling and warming, and during periods of equilibration.

The cooling/heating and pressure of the system can be integrated and controlled by a single controller utilizing temperature and pressure sensors. Alternatively, the temperature system can be controlled during cooling/warming by a single controller using one or more temperature sensors while the pressure generator operates separately using its own controller and sensor. The cooler/heater and pressure generator can each use their own sensor and controller. The preferred embodiment integrates all three components: heater, cooler, pressure generator into a single control, monitoring, and recording device. The entire high-pressure/low-temperature system's controls and monitoring devices can be automated using various means employing diverse equipment and methodologies.

### PREFERRED EMBODIMENT

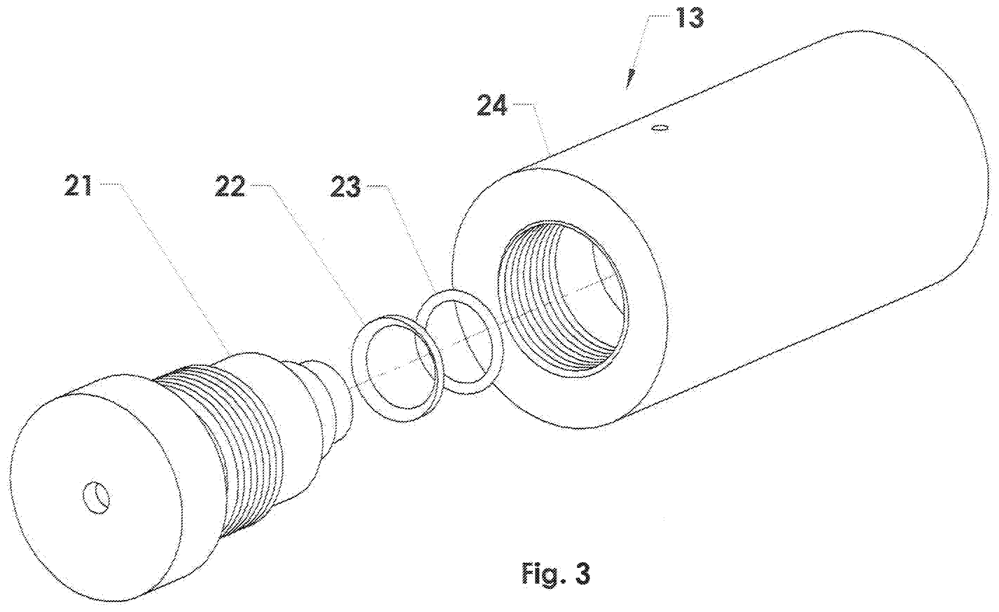

In the preferred embodiment of the invention (see FIG. 2), fluid (i.e. air) is used as the heat transfer medium. The device requires a vessel (FIG. 3) capable of containing pressures up to 276 MPa without failing; comprised of steel, stainless steel, titanium, or some other appropriate material, with a removable top, and a means of connecting the pressure generator to the vessel. The fluid-driven pressure generator can either be operated manually, using a separate timer to control the rate of pressurization and de-pressurization. Alternatively, the pressure generator can be driven mechanically, pneumatically, or hydraulically, or by other means, and controlled by an electrical, electronic, computer, or mechanical analog controller. Preferably, the pressure generator is mechanically driven and computer controlled.

The preferred means of connecting the pressure generator to the pressure vessel is by a system of pipes, valves, junctions, fittings, pressure gauge(s) and hydraulic fluid reservoir (FIG. 2). In order to decrease or increase temperature of the pressure vessel, a controlled cooling and heating system is required. In the case of a cooling/heating system using fluid as the medium for heat transfer, an insulated container is required to contain the cold/heat sink. A compressor and heat rejection unit can either be housed in the same container outside the cooling/warming device, or they can be in a separate enclosure and connected to the cooling device by insulated pipes.

The preferred embodiment of a mechanical refrigeration system employs a cylindrical reciprocating compressor with no power surge during start-up, and utilizes PID (Proportional-Integral-Derivative) controls. The heater can be separate from the evaporator with its own temperature sensor and temperature controller, or integrated with the evaporator, sharing the same controls. A temperature controller utilizing PID controls the refrigerator/heater by means of temperature data provided by a temperature sensor immersed in the heat transfer medium (fluid), or inserted in the pressure vessel. The preferred embodiment uses PID controls for temperature stability and RTD (Resistance Thermal Device) sensors for accuracy and precision.

The refrigeration system, using fluid as the heat transfer medium, has an evaporator as tall as the linear volume of the storage area of the pressure vessel, and a mixer to provide uniform temperature throughout the interior of the storage compartment. In the preferred embodiment the access is from above, by means of a removable insulated top, thus creating a cold well. The pressure vessel resides inside the storage compartment during cooling and heating, pressurization and de-pressurization.

A pressure gauge and a pressure transducer are used to monitor pressure. The pressure transducer is connected to a data acquisition system (DAQ) that is connected to a computer that displays and records the pressure. A thermistor is immersed in the cold well and a second thermistor is inserted into the pressure vessel. The data from these temperature sensors are transferred to a computer (via a DAQ as above), where they are displayed and recorded.

Tissue samples or organs are obtained immediately post-mortem, perfused according to accepted practice and bagged. Body heat is removed by submersing the bagged sample into a solution previously cooled to sub-zero temperature. The tissues and/or organ is then inserted into the pre-cooled pressure vessel filled with hydraulic fluid, the vessel is closed, air is removed, and the contents are pressurized and cooled. The items are held in cryostasis for a predetermined period or until needed. Recovery is accomplished by warming the pressure vessel followed by de-pressurization.

A laboratory prototype was used to validate the efficacy of the methodology and to determine rates of cooling and warming, pressurization and de-pressurization that are not deleterious to biological material. The benchtop device utilizes a PID controlled refrigeration system for controlled cooling of the vertical walls of an insulated enclosure. Said enclosure is open at the top and during operation the top is covered with insulation. The refrigeration system and controller are all housed in the same enclosure. Table 1 catalogs some of the materials stored, storage interval and post-storage condition.

The laboratory benchtop prototype device can be easily scaled up to accommodate entire organisms, such as humans for interplanetary or interstellar space travel. Some additional equipment may be necessary for the storage of organisms due to the weight of pressure vessels large enough to contain, but not limited to, a kidney, a heart, heart-lung or lung(s), a liver, a pancreas or other human or mammalian organs, either individually or in various combinations. An overhead winch or crane and/or a fork lift, or other weight-handling means, may be needed to move vessels and large, high-stability, walk-in or drive-in refrigerator(s) capable of holding temperatures as low as ˜22° C. will be required.

TABLE 1
Types of material preserved unfrozen using sub-zero ° C. storage at elevated pressure

| | Duration of | Storage | | | | |
|---|---|---|---|---|---|---|
| Material stored | storage | parameters T (° C.) | | Condition | | |
| (N) | (Solution) | Pressure (psi) | | after storage | Analysis used | Notes |
| Water | | −20° | C. | unfrozen | Ice nucleation | Theoretically water can |
| (N = 33) | | 30,000 | psi | | | be stored unfrozen |
| | | 207 | MPa | | | indefinitely at −20° C. & |
| | | | | | | 30,000 psi |
| Porcine kidney | 2 weeks | −18° | C. | Mean cell | PI/DAPI | No increase in cell |
| biopsies | (UW) | 30,000 | psi | viability = | Caspase | apoptosis post-storage |
| (N = 48) | | 207 | MPa | 94 ± 3% S.D. | Visual | No necrosis or |
| | | | | | | deterioration |
| Porcine kidney | 4 weeks | −18° | C. | Mean cell | I/DAPI | No necrosis or |
| biopsies | (UW) | 30,000 | psi | viability = | visual | deterioration, |
| (N = 12) | | 207 | MPa | 94 ± 3% S.D. | | No discoloration post- |
| | | | | | | storage |
| Porcine heart | 8 days | −8° | C. | 100% cell | PI/FDA | Organ not viable when |
| (N = 1) | (phosphate | 18,000 | psi | mortality | Visual | received, cut open |
| | buffer) | 124 | MPa | | | |
| Rabbit heart | 6 weeks | −18° | C. | ~90% cell | PI/FDA/DAPI | No necrosis or |
| (N = 3) | (phosphate | 30,000 | psi | viability | visual | deterioration, |
| | buffer) | 207 | MPa | | | No discoloration post- |
| | | | | | | storage |
| Rabbit heart | 7 days | −18° | C. | ~90% cell | PI/FDA/DAPI | No necrosis or |
| (N = 16) | (phosphate | 30,000 | psi | viability | visual | deterioration, |
| | buffer) | 207 | MPa | | | No discoloration post- |
| | | | | | | storage |
| Rabbit kidney | 10 days | −18° | C. | ~90% cell | PI/FDA/DAPI | No necrosis or |
| (N = 12) | (CryoStasis) | 30,000 | psi | viability | visual | deterioration, |
| | | 207 | MPa | | | No discoloration post- |
| | | | | | | storage |
| Rat heart | 7 days | −18° | C. | ~90% cell | PI/FDA | Good condition |
| (N = 2) | (CryoStasis) | 30,000 | psi | viability | Visual | No necrosis |
| | | 207 | MPa | | Langendorf | No discoloration |
| Rat kidney | 8 days | −20° | C. | ~90% cell | PI/FDA | Organs intact, |
| (N = 4) | (CryoStasis) | 30,000 | psi | viability | Visual | unchanged compared to |
| | | 207 | MPa | | Morphology | before storage |
| Bovine | 7 days | −18° | C. | 30% cell | PI/FDA/DAPI | Air bubbles problem |
| spermatozoa | (Tolga) | 30,000 | psi | viability | MTT assay | |
| (N = 3 × 300,000) | | 207 | MPa | | motility | |
| Oyster larvae | 23 days | −18° | C. | 5-20% | PI/FDA | Survival was age- |
| (N = 6 cohorts) | (sea water) | 30,000 | psi | viability | Morphological | dependent; best in 3- |
| | | 207 | MPa | | Motility | week veliger larvae |
| Mud minnows | 7 days | −18° | C. | None survived | Visual | Fish intact, no damage |
| (N = 7) | (water) | 30,000 | psi | but unchanged | Morphological | Could not function when |
| | | 207 | MPa | | Physiological | returned to aquarium |
| HEK293 cells | 12 hours | 20° | C. | 5% cell | PI/FDA | Poor survival at 20° C. |
| (N = 3 × 100,000) | (DMEM) | 15,000 | psi | viability | MTT assay | and high pressure |
| | | 104 | MPa | | | |
| Mitochondria | 1 day- | −18° | C. | Always intact | DAPI | Intact in all studies |
| (various sources) | 1 month | 30,000 | psi | Function | MTT assay | Function confirmed in |
| | (various | 207 | MPa | confirmed in | | some studies |
| | media) | | | some trials | | |
| Catalase | 3 days | −18° | C. | Function | H₂O₂ | Test of high pressure |
| (N = 2) | (water) | 30,000 | psi | confirmed | | effect on enzymes |
| | | 207 | MPa | | | |
| PEG | 1 day- | −18° | C. | No change | Osmometry | FPD unchanged |
| (N = 100+) | 1 month | 30,000 | psi | | | |
| | | 207 | MPa | | | |
| EG | 1 day- | −18° | C. | No change | Osmometry | FPD unchanged |
| (N = 12) | 1 month | 30,000 | psi | | | |
| | | 207 | MPa | | | |
| Phosphate buffer | 1 day- | −18° | C. | No change | Cell survival | Cells survived |
| (N = 16) | 1 month | 30,000 | psi | | | |
| | | 207 | MPa | | | |
| DMEM | 12 hours | 20° | C. | No change | Cell survival | Cells survived |
| (N = 3) | (DMEM) | 15,000 | psi | | | |
| | | 104 | MPa | | | |
| U. of Wisconsin | 1 day- | −18° | C. | No change | Cell survival | Cells survived |
| Solution | 1 month | 30,000 | psi | | | |
| (N = 60) | | 207 | MPa | | | |
| CryoStasis | 1 day- | −18° | C. | No change | Osmometry | FPD unchanged |
| Solution | 1 month | 30,000 | psi | | | AFP activity unchanged |
| (N = 22) | | 207 | MPa | | | |
| Antifreeze | 3 days | −18° | C. | No change | Osmometry | FPD unchanged |
| Proteins | (CryoStasis) | 30,000 | psi | | | |
| (N = 4) | | 207 | MPa | | | |
| Bacteria & algae | 23 days | −18° | C. | Cells intact & | Microscopic | Cells intact & motile |
| (N = 6) | (sea water) | 30,000 | psi | motile | | |
| | | 207 | MPa | | | |
| Sea water | 23 days | −18° | C. | No change | Osmometry | FPD unchanged |
| (N = 6) | | 30,000 | psi | | Larval | |
| | | 207 | MPa | | survival | |

Acronyms: PI (Propidium Iodide); FDA (Fluorescein Diacetate); DAPI (4′,6-diamidino-2-phenylindole); DMEM (Dulbecco Modified Eagle Medium); PEG (Propylene Glycol); EG (Ethylene Glycol); FPD (Freezing Point Depression)

We claim:

1. A method for storing/preservation, including but not limited to, water, organic and inorganic aqueous-based materials/substances/media, materials in aqueous suspension, aqueous solutions, aqueous mixtures, aqueous colloids, aqueous-based materials, biological materials, biologics, and materials of biological origin at temperatures below their freezing, i.e. melting, temperature at ambient pressure by means of increased pressure. Increasing the pressure applied to any or all of the above materials, in a container, depresses their freezing, i.e. melting temperature (point). The temperature range for storage where it is not possible for the above substances to freeze or vitrify extends from −0.001° C. to −21.985° C. The melting point, i.e. freezing point, of the above materials being depressed by pressure over the pressure range from ambient pressure to 209.9 MPa. These biological materials are, but not limited to, organic molecules and molecular complexes, nucleic acids, saccharides, amino acids, peptides, proteins, enzymes, organelles, cells, tissues, organs, and organisms.

2. A method of storing aqueous-based material under pressure to prevent phase transition to solid and maintain it in stable liquid state according to claim 1, wherein the material stored is water.

3. A method of storing aqueous-based material under pressure to prevent phase transition to solid and maintain it in stable liquid state according to claim 1, wherein the material stored is water containing inorganic solutes in aqueous solution.

4. A method of storing aqueous-based material under pressure to prevent phase transition to solid and maintain it in stable liquid state according to claim 1, wherein the material stored is water containing organic solutes in aqueous solution.

5. A method of storing aqueous-based material under pressure to prevent phase transition to solid and maintain it in stable liquid state according to claim 1, wherein the material stored is water containing organic and inorganic solutes in aqueous solution.

6. A method of storing aqueous-based material under pressure to prevent phase transition to solid and maintain it in stable liquid state according to claim 1, wherein the material stored is water and a mixture of organic material.

7. A method of storing aqueous-based material under pressure to prevent phase transition to solid and maintain it in stable liquid state according to claim 1, wherein the material stored is water containing a colloid(s).

8. A method of storing aqueous-based material under pressure to prevent phase transition to solid and maintain it in stable liquid state according to claim 1, wherein the material stored is water in a mixture with either or both organic and/or inorganic materials.

9. A method of storing aqueous-based material under pressure to prevent phase transition to solid and maintain it in stable liquid state according to claim 1, wherein the material stored is water in a mixture with biological material(s).

10. A method of storing aqueous-based material under pressure to prevent phase transition to solid and maintain it in stable liquid state according to claim 1, wherein the material stored is water with biological material(s) present and/or in suspension.

11. A method of storing aqueous-based material under pressure to prevent phase transition to solid and maintain it in stable liquid state according to claim 1, wherein the material stored is water containing organic and/or inorganic solutes and with biological material(s) present and/or in suspension.

12. A method of storing aqueous-based material under pressure to prevent phase transition to solid and maintain it in stable liquid state according to claim 1, wherein the material stored is water containing organic and/or inorganic solutes and colloid(s) with biological material(s) present and/or in suspension.

13. A method of storing aqueous-based material under pressure to prevent phase transition to solid and maintain it in stable liquid state according to claim 1, wherein the material stored is water in a mixture with compounds, organic and/or inorganic, and containing solutes both organic and/or inorganic, colloid(s), with biological material(s) present and/or in suspension.

14. A method of depressing the supercooling temperature (point) of, but not limited to, organic and inorganic aqueous-based materials/substances/media, materials in aqueous suspension, aqueous solutions, aqueous mixtures, aqueous colloids, aqueous-based materials, biological materials, and materials of biological origin at temperatures below their freezing, i.e. melting, temperature at ambient pressure by means of increasing the pressure applied to said material/substance and cooling to temperature(s) below their freezing/melting point at a given pressure. By these means said materials/substances can be supercooled and remain in a metastable liquid state over the range from −0.001° C. to −92° C. Said supercooling occurs over the pressure range from ambient pressure to 209.9 MPa. The material being stored is supercooled, if the storage temperature is below the pressure-depressed (pressure-determined) freezing/melting point of said material above. Above described biological materials are, but not limited to, organic molecules and molecular complexes, nucleic acids, saccharides, amino acids, peptides, proteins, enzymes, biologics, organelles, cells, tissues, organs, and organisms.

15. A method of storing aqueous-based material under pressure to prevent phase transition to solid and maintain it in metastable supercooled liquid state as in claim 14, wherein the material stored is water.

16. A method of storing aqueous-based material under pressure to prevent phase transition to solid and maintain it in metastable supercooled liquid state as in claim 14, wherein the material stored is water containing inorganic solutes.

17. A method of storing aqueous-based material under pressure to prevent phase transition to solid and maintain it in metastable supercooled liquid state as in claim 14, wherein the material stored is water containing organic solutes.

18. A method of storing aqueous-based material under pressure to prevent phase transition to solid and maintain it in metastable supercooled liquid state as in claim 14, wherein the material stored is water containing organic and/or inorganic solutes.

19. A method of storing aqueous-based material under pressure to prevent phase transition to solid and maintain it in metastable supercooled liquid state as in claim 14, wherein the material stored is water and a mixture of organic material.

20. A method of storing aqueous-based material under pressure to prevent phase transition to solid and maintain it in metastable supercooled liquid state as in claim 14, wherein the material stored is water containing colloids.

21. A method of storing aqueous-based material under pressure to prevent phase transition to solid and maintain it in metastable supercooled liquid state as in claim 14, wherein the material stored is water in a mixture with either or both organic and/or inorganic materials.

22. A method of storing aqueous-based material under pressure to prevent phase transition to solid and maintain it in metastable supercooled liquid state as in claim 14, wherein the material stored is water in a mixture with biological material(s) and/or other compounds.

23. A method of storing aqueous-based material under pressure to prevent phase transition to solid and maintain it in metastable supercooled liquid state as in claim 14, wherein the material stored is water with biological material(s) present and/or in suspension.

24. A method of storing aqueous-based material under pressure to prevent phase transition to solid and maintain it in metastable supercooled liquid state as in claim 14, wherein the material stored is water containing organic and/or inorganic solutes and with biological material(s) present and/or in suspension.

25. A method of storing aqueous-based material under pressure to prevent phase transition to solid and maintain it in metastable supercooled liquid state as in claim 14, wherein the material stored is water containing organic and/or inorganic solutes, and colloid(s) with biological material(s) present and/or in suspension.

26. A method of storing aqueous-based material under pressure to prevent phase transition to solid and maintain it in metastable supercooled liquid state as in claim 14, wherein the material stored is water in a mixture with compounds, organic and/or inorganic, and containing solutes both organic and/or inorganic, colloid(s), with biological material(s) present and/or in suspension.

27. A method for lowering the freezing point of above said materials stored under the conditions described in claims 1 and 14 by further depressing the freezing temperature of said aqueous media by non-colligative means. These non-colligative substances are, but not limited to, antifreeze proteins, ice binding proteins, antifreeze saccharides, ice binding saccharides, ice binding peptides, and other non-colligative agents that provide an additive freezing point depression by means of ice inhibiting or ice binding, thus preventing, inhibiting, controlling, and/or sequestering ice crystal growth, and/or preventing nucleation of ice. Above said biological materials stored are, but not limited to, amino acids, peptides, proteins, enzymes, biologics, organelles, cells, tissues, organs, and organisms.

28. A method for lowering the freezing point of the said materials and conditions described in claims 1, 14 and 27 by further depressing the freezing temperature of the materials by the addition of solutes to the media and material being stored, resulting in a further freezing point depression of 1.86° C. per mole of solute added; or a fraction or multiplier thereof. Freezing point is depressed by 1.86° C. per mole or fraction of 1.86° C. per mole fraction of solute added. The material added must be soluble in water. Above said biological materials stored are, but not limited to, organic molecules and molecular complexes, nucleic acids, saccharides, organic molecules and molecular complexes, nucleic acids, saccharides, amino acids, peptides, proteins, enzymes, biologics, organelles, cells, tissues, organisms.

29. A method for lowering the freezing point of above said aqueous media under the conditions described above in claims 1 through 28, inclusive, by further depressing the freezing temperature of said aqueous media by colligative means of adding a mole or mole fraction of a solute or solutes to the aqueous solution, mixture, colloid or combination thereof. Then, a further freezing point depression resulting from the addition of non-colligative, claim 27, substances, including but not limited to, antifreeze proteins, antifreeze saccharides, ice binding peptides, and other non-colligative agents that provide an additive freezing point depression by means of ice inhibiting or ice binding, thus preventing, inhibiting, controlling, and/or sequestering ice crystal growth. Said media may or may not contain biological material, including but not limited to, organic molecules and molecular complexes, nucleic acids, saccharides, amino acids, peptides, proteins, enzymes, biologics, organelles, cells, tissues, organisms.

30. All of the hereto described herein, named and elucidated methods, materials, and properties can/are being used and/or incorporated in total, concert, partially, or individually to depress the temperature at which water, with or without additives remains in liquid state.

31. A device for storing any or all of the above material(s)/substance(s) under pressure and at depressed temperature including refrigeration/heating system(s). Said device consisting of, but not limited to, a pressure vessel with an interior volume, vessel walls, and an attachable/detachable top, all capable of withstanding interior pressures up to and in excess of, but not limited to, a storage pressure of ˜210 MPa. A refrigeration system capable of lowering the temperature of the vessel and contents to, but not limited to, −22° C. for stable liquid state storage, and/or −92° C. for supercooled storage.

32. Said pressure vessel, claim 31, is pressurized and de-pressurized by means of, but not limited to, a pressure generator that generates pressure either pneumatically, or hydraulically, or mechanically by means of, but not limited to, a manual drive, or a mechanical drive, or a pneumatic drive, or a hydraulic drive that is controlled either manually, mechanically, electrically, electronically or by computer.

33. Said pressure vessel, claim 31, is pressurized by a pneumatic pressure generator driven manually.

34. Said pressure vessel, claim 31, is pressurized by a pneumatic pressure generator driven mechanically.

35. Said pressure vessel, claim 31, is pressurized by a pneumatic pressure generator driven hydraulically.

36. Said pressure vessel, claim 31, is pressurized by a pneumatic pressure generator driven pneumatically.

37. Said pressure vessel, claim 31, is pressurized by a hydraulic pressure generator driven manually.

38. Said pressure vessel, claim 31, is pressurized by a hydraulic pressure generator driven mechanically.

39. Said pressure vessel, claim 31, is pressurized by a hydraulic pressure generator driven pneumatically.

40. Said pressure vessel, claim 31, is pressurized by a hydraulic pressure generator driven hydraulically.

41. Said pressure generator's, claim 32, rate of pressurization and de-pressurization of the pressure vessel is controlled either mechanically, manually, electrically, electronically, or using a computer.

42. Said pressure generator's, claim 32, rate of pressurization and de-pressurization of the pressure vessel is controlled mechanically.

43. Said pressure generator's, claim 32, rate of pressurization and de-pressurization of the pressure vessel is controlled manually.

44. Said pressure generator's rate, claim 32, of pressurization and de-pressurization of the pressure vessel is controlled electrically.

45. Said pressure generator's, claim 32, rate of pressurization and de-pressurization of the pressure vessel is controlled electronically.

46. Said pressure generator's, claim 32, rate of pressurization and de-pressurization of the pressure vessel is controlled by means of a computer.

47. Said pressure vessel, claim 31, is attached to the pressure generator, claim 32, by a system of pipes and piping components, including but not limited to: valves, tees, unions, collars, glands, 4-way crosses, pressure gauge(s), pressure transducer(s), thermal well(s), temperature sensors, and a source of pressurization fluid.

48. Said system, claim 47, of pipes and piping components, including but not limited to: pipes.

49. Said system, claim 47, of pipes and piping components, including but not limited to: manually operated valves.

50. Said system, claim 47, of pipes and piping components, including but not limited to: solenoid operated valve.

51. Said system, claim 47, of pipes and piping components, including but not limited to: valves operated by motors.

52. Said system, claim 47, of pipes and piping components, including but not limited to: pressure controlled valves.

53. Said system, claim 47, of pipes and piping components, including but not limited to: computer controlled valves.

54. Said system, claim 47, of pipes and piping components, including but not limited to: piping tees.

55. Said system, claim 47, of pipes and piping components, including but not limited to: piping four-way connectors.

56. Said system, claim 47, of pipes and piping components, including but not limited to: piping unions.

57. Said system, claim 47, of pipes and piping components, including but not limited to: piping collars.

58. Said system, claim 47, of pipes and piping components, including but not limited to: piping collars with or without glands.

59. Said system, claim 47, of pipes and piping components, including but not limited to: pressure gauge(s).

60. Said system, claim 47, of pipes and piping components, including but not limited to: pressure transducer(s).

61. Said system, claim 47, of pipes and piping components, including but not limited to: thermal wells.

62. Said system, claim 47, of pipes and piping components, including but not limited to: temperature sensors.

63. Said system, claim 47, of pipes and piping components, including but not limited to: drive/pressurization fluid reservoir(s).

64. A device, claim 31, that has, but is not limited to, a refrigeration/heating system for cooling and/or heating a fluid, in a chamber containing a pressure vessel(s), or that flows through a series of circuits in the pressure vessel's wall(s) or is attached to the outside of a pressure vessel(s) during or after pressurization; and warms said fluid while warming the pressure vessel during or after de-pressurization. The refrigerator/heater is one of, but not limited to, the following devices or configurations or combination(s) thereof. Where said refrigerator and heater are separate components that are controlled either manually, electrically, electronically, or by means of a computer. Where said refrigerator and heater are integrated into one component that is controlled either manually, electrically, electronically, or by means of a computer. Where said refrigerator uses reverse cycle for heating and is controlled either manually, electrically, electronically, or by means of a computer. Where the refrigerator and/or heater uses a piston compressor, evaporator, and condenser. Where the refrigerator and/or heater uses a reciprocating piston compressor, evaporator, and condenser, and is controlled either manually, electrically, electronically, or by means of a computer. Where the refrigerator/heater is thermoelectric and is controlled either manually, electrically, electronically, or by means of a computer. Where the refrigerator/heater is a sterling refrigerator, sterling pulse tube cooler, and/or heater and is controlled either manually, electrically, electronically, or by means of a computer. Where the refrigerator/heater is a sonic or ultrasonic device and is controlled either manually, electrically, electronically, or by means of a computer. Where the refrigerator operates by means of evaporative cooling (e.g. liquid nitrogen, dry ice) and is controlled either manually, electrically, electronically, or by means of a computer. Where heating and cooling are by radiation and are controlled either manually, electrically, electronically, or by means of a computer. Where heating and cooling are by convection and are controlled either manually, electrically, electronically, or by means of a computer. Where heating and cooling are by induction and are controlled either manually, electrically, electronically, or by means of a computer. Where resistance is used for heating and is controlled either manually, electrically, electronically, or by means of a computer. Where lasers or masers are used for heating and/or cooling and are controlled either manually, electrically, electronically, or by means of a computer.

65. Where said refrigerator, claim 64, and heater are separate components that are controlled either manually, electrically, electronically, or by means of a computer.

66. Where said refrigerator, claim 64, and heater are integrated into one component that is controlled either manually, electrically, electronically, or by means of a computer.

67. Where said refrigerator, claim 64, uses reverse cycle for heating and is controlled either manually, electrically, electronically, or by means of a computer.

68. Where the refrigerator, claim 64, and/or heater uses a piston compressor, evaporator, and condenser.

69. Where the refrigerator, claim 64, and/or heater uses a reciprocating piston compressor, evaporator, and condenser, and is controlled either manually, electrically, electronically, or by means of a computer.

70. Where the refrigerator/heater, claim 64, is thermoelectric and is controlled either manually, electrically, electronically, or by means of a computer.

71. Where the refrigerator/heater, claim 64, is a sterling refrigerator, sterling pulse tube cooler, and/or heater and is controlled either manually, electrically, electronically, or by means of a computer.

72. Where the refrigerator/heater, claim 64, is a sonic or ultrasonic device and is controlled either manually, electrically, electronically, or by means of a computer.

73. Where the refrigerator, claim 64, operates by means of evaporative cooling (e.g. liquid nitrogen, dry ice) and is controlled either manually, electrically, electronically, or by means of a computer.

74. Where heating and cooling, claim 64, are by radiation and are controlled either manually, electrically, electronically, or by means of a computer.

75. Where heating and cooling, claim 64, are by convection and are controlled either manually, electrically, electronically, or by means of a computer.

76. Where heating and cooling, claim 64, are by induction and are controlled either manually, electrically, electronically, or by means of a computer.

77. Where resistance is used for heating, claim 64, and is controlled either manually, electrically, electronically, or by means of a computer.

78. Where lasers or masers are used for heating and/or cooling, claim 64, and are controlled either manually, electrically, electronically, or by means of a computer.

79. A device, claim 31, with, but not limited to, control(s), a set of controls, a control system or systems to initiate and/or maintain, or stop its operation; and to set and/or adjust the environment within the system as a whole and its components. The temperature inside the pressure vessel can be cooled or maintained by a cooling system with a temperature controller, and the temperature inside the pressure vessel can be warmed or maintained by a heating system using a separate controller. The controller for cooling and the controller for warming can be operated simultaneously. A single temperature controller can be used to control the temperature during cooling and warming. The temperature controller used during cooling can control the rate of temperature change. The temperature controller used during warming can control the rate of temperature change. A single controller can be used to control cooling and warming and the rate of cooling and warming. A separate controller can be used during pressurization to control the rate of pressurization or pressurize ballistically. An additional controller can be used during pressurization to control the rate of de-pressurization or de-pressurize ballistically. A single controller can be used to control pressurization and de-pressurization and the rate of pressurization and de-pressurization. A single controller can be used to control temperature during warming and cooling, and the rate thereof; it can also control pressurization and de-pressurization, and the rate thereof. Any or all of the aforesaid control devices both for pressure and for temperature, or individually, can be mechanical, electrical, electronic, or computer. Any or all of these control devices can control by means of set point, rate of change, duration at set point for either or both temperature and pressure. Said controller(s) have a temperature sensor that provides the controller with the current temperature inside the refrigerator and/or pressure vessel. Said controller(s) have a pressure sensor, transducer, and/or gauge that provides the controller with the current pressure inside the pressure vessel, piping system or parts thereof.

80. Control(s), claim 79, such that the control(s) can operate and be independent for temperature inside the pressure vessel while cooling from a different control(s) can operate during warming.

81. Control(s), claim 79, comprised of a single temperature controller can operate to control the temperature during cooling and warming.

82. Control(s), claim 79, such that the temperature controller used during cooling can control the rate of temperature change.

83. Control(s), claim 78, such that the temperature controller used during warming can control the rate of temperature change.

84. Control(s), claim 79, such that a single controller can be used to control cooling and warming and the rate of cooling and warming.

85. Control(s), claim 79, such that a separate controller can be used during pressurization to control the rate of pressurization or pressurize ballistically.

86. Control(s), claim 79, such that a separate additional controller can be used during pressurization to control the rate of de-pressurization or de-pressurize ballistically.

87. Control(s), claim 79, such that a single controller can be used to control pressurization and de-pressurization and the rate of pressurization and de-pressurization.

88. Control(s), claim 79, such that a single controller can be used to control temperature during warming and cooling and the rate thereof; it can also control pressurization and de pressurization, and the rate thereof.

89. Control(s), claim 79, such that any or all of the aforesaid control devices, both for pressure and for temperature, or individually for temperature and/or pressure, for heating and/or cooling, for pressurization and/or de-pressurization, can be mechanical, electrical, electronic, or computer.

90. Control(s), claim 79, such that any or all of these control devices can control by means of set point, rate of change, duration at set point, for either or both, temperature and pressure. Said controller(s) have a temperature sensor that provides the controller with the current temperature inside the refrigerator and/or pressure vessel. Said controller(s) have a pressure sensor, transducer, and/or gauge that provides the controller with the current pressure inside the pressure vessel, piping system or parts thereof.

91. A device, claim 31, that has the means for monitoring temperature, by reading and/or recording the temperature inside the refrigerator/heater, inside the pressure vessel, inside the wall of the pressure vessel, or from the surface of the pressure vessel, in real time. Temperature readings, either analog or digital, can be taken automatically at intervals, or manually at intervals, said readings can be recorded manually, mechanically, electrically, electronically, or my means of computer(s). Temperature readings are provided by means of thermometer(s), thermistor(s), resistance thermal device(s) (RTD), thermocouple(s), infra-red sensor(s), infra-red camera(s), pyrometer(s), spring thermometer(s), liquid in a column thermometer(s), or any other mechanical, chemical, liquid crystal, electrical, or electronic sensor(s). These data from any and/or all of the temperature sensors, listed above, can be used as input temperature information for the control(s) in claim 79, above.

92. A device, claim 31, that has a means for monitoring pressure, by reading and/or recording pressure inside the pressure vessel, and/or in or from the pressure generator, and/or inside part(s) or all of the piping system. Pressure readings are produced from pressure transducer(s), analog pressure gauge(s), and displayed in real time on analog and/or digital gauge(s). Data from the pressure gauge(s) or pressure transducer(s) can are recorded mechanically, electrically, electronically, or using computer(s). These data from any and/or all pressure sensors, listed above, can be used as pressure information for the control(s) in claim 79, above.
